(19) United States
(12) Patent Application Publication
(10) Pub. No.: US 2010/0106014 A1
(43) Pub. Date: Apr. 29, 2010

(54) APPARATUS FOR THE SPATIAL LOCALIZATION OF A MOVEABLE BODY PART

(75) Inventors: Thomas Broennimann, Wolfisberg (CH); Laurent Cloutot, Killwangen (CH)

Correspondence Address: LERNER GREENBERG STEMER LLP, P O BOX 2480, HOLLYWOOD, FL, 33022-2480, US

(73) Assignee: SIEMENS SCHWEIZ AG, Zurich (CH)

(21) Appl. No.: 12/525,954

(22) PCT Filed: Feb. 6, 2008
(86) PCT No.: PCT/EP08/00902

§ 371 Date: Aug. 5, 2009

(30) Foreign Application Priority Data
Feb. 6, 2007 (EP) 07 002 473.2

(51) Int. Cl. A61B 6/00; G01J 1/58

(52) U.S. Cl. 600/431; 250/458.1

(57) ABSTRACT

An apparatus for the spatial localization of a moveable body part, in which the body part is situated inside a movement volume on the surface extending as far as to the inside of a living being. The apparatus includes: at least one optical recording apparatus outside the being, at least one meterable fluorophore which can be introduced in the region of the body part, an extrinsic radiation source which is arranged outside the being and from which radiation propagates in the direction of the movement volume, by way of which spectral excitation of the fluorophore takes place in that a wave emitted by the fluorophore is produced and can be determined at least at a wavelength which can be measured by the optical recording apparatus, the optical recording apparatus has at least one optical axis which can be oriented in the direction of the body part and the movement volume thereof, the optical recording apparatus has at least one optoelectric transducer which is perpendicular to the optical axis and outputs an output signal from which a distance between the fluorophore and a reference point can be determined.

The invention relates to an apparatus for the spatial localization of a moveable body part as claimed in the preamble of claim 1.

During radiotherapy of a tumor by means, for example, of gamma radiation or proton radiation or when imaging a tumor inside the body of a being, numerous methods or apparatuses exist in which it is desired to ensure a three-dimensional localization of the tumor or, more generally, of a moveable body part, which is as accurate as possible and which may be reproduced. Some methods (“in vivo” imaging methods, for example, by means of radiography using X-rays) are, however, classified as harmful “invasive” methods and should therefore only be used in a restricted manner. Less harmful “in vivo” methods, such as for example imaging methods using magnetic resonance, in addition to their complexity, their extra work and their cost remain difficult to combine simultaneously with radiation treatment. Other (“ex vivo”) methods use (for example optical) detectable features/markings, which may be applied/incorporated on the “surface” of a patient relative to a known coordinate system and are identifiable. However, these methods lack accuracy and also reproducibility, as physiological changes to the patient (for example weight loss) or unavoidable (at worst unexpected) movements (for example by respiration, by trembling, etc.) during treatments and/or repositioning of the patient/body parts, occur between treatments. Furthermore, methods/apparatuses exist for holding the patient or a body part on, for example, a support table during the radiotherapy or devices for measuring respiration, but, on the one hand, these solutions reduce the “comfort” of the patient and/or do not allow spatial determination/localization of a tumor in a known coordinate system which is 100% accurate.

Recently, however, a tumor inside a small animal (mouse) was able to be imaged by means of a CCD camera arranged externally to the mouse. In this connection, a fluorophore (hematoporphyrin) was injected into the bloodstream of the mouse, which is able to accumulate selectively in tumors due to a tumor affinity. Additionally, light from an extrinsic light source (i.e. arranged outside the mouse) in the red/infrared spectral wavelength range illuminates the mouse or at least a part of the mouse comprising the tumor. As the emitted light is able to penetrate soft tissue parts of the mouse as far as the location of the fluorophore and the fluorophore had been accordingly metered, an “autofluorescence emission” light signal from the excited fluorophore was able to be obtained and measured by the CCD camera as reflection and/or backscatter. Thus it has become possible to produce an “in vivo” intensity image of the tumor with a very small risk and/or no risk of injury. According to the features of the image of the tumor or by using suitable filters, therefore, statements relating to the shape or type of the unhealthy cells may be made. Such a method is disclosed in more detail in the citation: L. Celentano, P. Laccetti, R. Liuzzi, G. Mettivier, M. C. Montesi, M. Autiero, P. Riccio, G. Roberti, P. Russo, Member, IEEE and M. Salvatore, “Preliminary Tests of a Prototype System for Optical and Radionuclide Imaging in Small Animals”, IEEE TRANSACTIONS ON NUCLEAR SCIENCE, VOL. 50, No. 5, October 2003, pages 1693-1701. This attractive “in vivo” observation method, which is advantageous as it is not significantly harmful, is very well suited to small animals, but at the same time no indications about an accurate, spatial (for example metric) localization of a moveable tumor are provided, as the current method allows a purely two-dimensional “planar imaging” of autofluorescing tumors.

The object underlying the invention is to provide an apparatus for accurate spatial localization of a moveable body part (tumor, carcinoma, etc.) of a living being (i.e. also a human patient).

Moreover, the apparatus is intended to be designed so that a potential risk from intensive (i.e. invasive) irradiation of the patient is avoided. Similarly, during radiotherapy of a patient the apparatus should also be able to localize “in vivo” as far as possible in real time and three-dimensionally, in order to destroy a tumor or carcinoma for example.

According to the invention the object is achieved by the features of claim 1.

An apparatus for the spatial (one-, two- or three-dimensional) localization of a moveable body part is proposed, in which the body part (for example a tumor) is situated inside a movement volume on the surface extending as far as the inside of a living being (for example of a person). The apparatus according to the invention has the following components:
at least one optical recording apparatus arranged outside the being,
at least one meterable fluorophore which can be introduced in the region of the body part,
an extrinsic radiation source which is arranged outside the being and from which radiation propagates in the direction of the movement volume, as a result of which spectral excitation of the fluorophore takes place, by a wave emitted by the fluorophore being produced and at least one wavelength which can be measured by the optical recording apparatus being determinable,
the optical recording apparatus has at least one optical axis which can be orientated in the direction of the body part and the movement volume thereof,
the optical recording apparatus has at least one optoelectric transducer which is arranged at right angles to the optical axis and outputs an output signal from which a distance between the fluorophore and a reference point can be determined. The reference point may, if desired, be selected in an absolute spatial coordinate system. A known “isocenter” of an adjacent, therapeutic irradiation unit may be taken as a preferred reference point. The reference point may also form, for example, a separate point of intersection on the optical axis within the movement volume.

The apparatus according to the invention is very well suited to different degrees of one-, two- or three-dimensional localization, depending on, amongst other things, how may optoelectric transducers are used. This aspect is disclosed in more detail below.

Generally, the invention may be implemented with a plurality of embodiments of interest, in which;
the optoelectric transducer of the optical recording apparatus is a photodiode or a group of adjacent photodiodes or pixels, as preferably in a camera,
the optical axis of the photodiode or of the camera can be moved, preferably by means of pivoting or rotation, such that the optical axes formed thereby form a positive angle with one another and form a separate point of intersection in the movement volume.

### Or:

the optical recording apparatus comprises a plurality of photodiodes or a plurality of cameras with integrated adjacent optoelectric transducers (for example in a planar manner on a “chip”), the optical axes thereof forming a positive angle with one another and forming a separate point of intersection (i.e. an actual isocenter) in the movement volume.

The output signal(s) from the optical recording apparatus change(s) as a result of the actual isocenter being moveable by the movement of the body part. The deviations of the output signal formed and determined thereby therefore allow, with a high degree of accuracy, a spatial dynamic tracking of the body part (i.e. of the fluorophore), which takes place relative to the reference point (for example a reference isocenter of an irradiation unit). Subsequently attempts are made to ensure that newly occurring deviations are continuously determined relative to a previously localized actual isocenter (and that said deviations are compensated optionally by a triggered positioning means and the actual isocenter from the detected fluorophore is continuously retained as the next reference isocenter) so that the body part can always be detected by the optical recording apparatus.

In this connection, two measuring signals may inter alia be determined for instance from a fluorescence reflected from the fluorophore, said measuring signals being recorded from two directions oriented obliquely to one another. As both directions in a known spatial coordinate system are known, two- and/or three-dimensional coordinates relating to the position of the fluorophore may be accurately spatially determined from both measuring signals (for example via a propagation time measurement or via a recording of a two-dimensional digitalized intensity image of the backscattered fluorescence).

At the appropriate a collimating radiation direction of the extrinsic light source is located centrally between the directions of optical axes of the optical recording apparatus. Further light radiation may also be used for the extrinsic light source, such as for example an annular source of illumination, which is positioned above the region to be measured. Naturally, more than one light source or more than two optical axes may be used for the optical recording apparatus. Thus the accuracy or the speed is increased and possible light-absorbing locations may be more powerfully illuminated, in particular in order to reach a tumor located deep in the body. The apparatus according to the invention may function in real time and instantly provide 3D coordinates of a tumor, for example more than 50 recordings per second. This measurement is dependent on the technology of the optical recording apparatus used and on the amount of light of the back-radiating fluorescence (and therefore depends on the dosage rate of the fluorophore as well as on the efficiency of the extrinsic light source). Should the recorded measuring signals be too weak, and/or have signal-noise ratios which are too high, several temporally separated measuring signals may be implemented at each optical axis, which are simply added together statistically depending on the intensity and/or averaged. Thus, advantageously the noise values of the recorded images are also averaged, although at the expense of the recording speed. Other fluorescing signal wavelengths which are back-radiated to the optical recording apparatus as those of the fluorophor are also optically filtered. Ideally, the back-radiation of the fluorophore has at least one wavelength outside the spectral range of the extrinsic light source, so that no undesired light of the light source is recorded on the optical recording apparatus but only the single light which is emitted by the fluorophore and which is also able to be filtered. It is also avoided for interference reasons that a component (light source, optical axis of the optical recording apparatus) of the apparatus according to the invention is located in or along a/the beam path or paths of an irradiation unit (using for example gamma or proton radiation). On the contrary, the components are positioned furthest away from the therapeutic radiation.

Advantageous embodiments of the invention are set forth in the sub-claims.

The invention is explained hereinafter in an exemplary embodiment, with reference to the drawings.

in which;

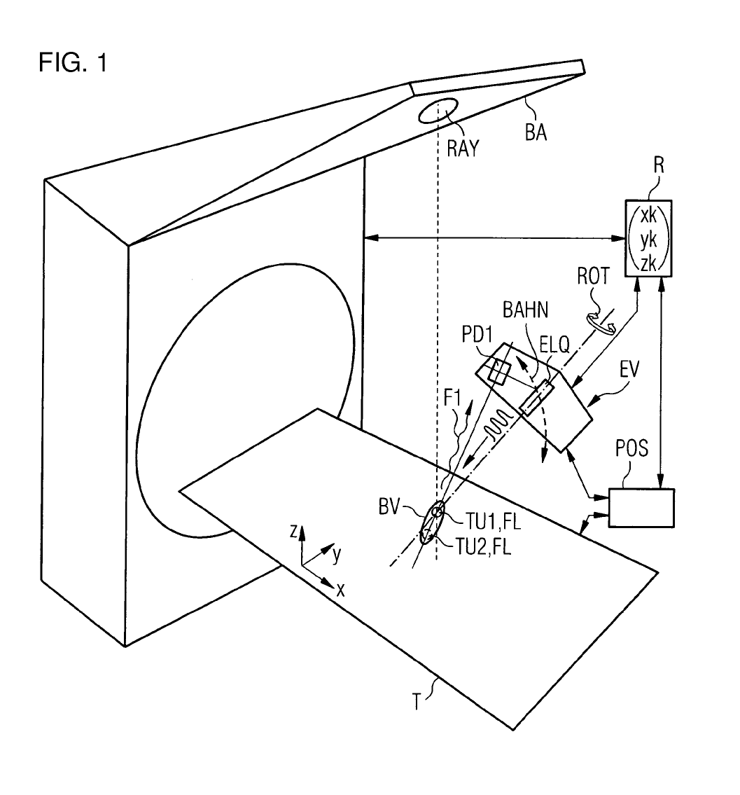

FIG. 1 shows a first apparatus according to the invention in a radiotherapeutic irradiation unit,

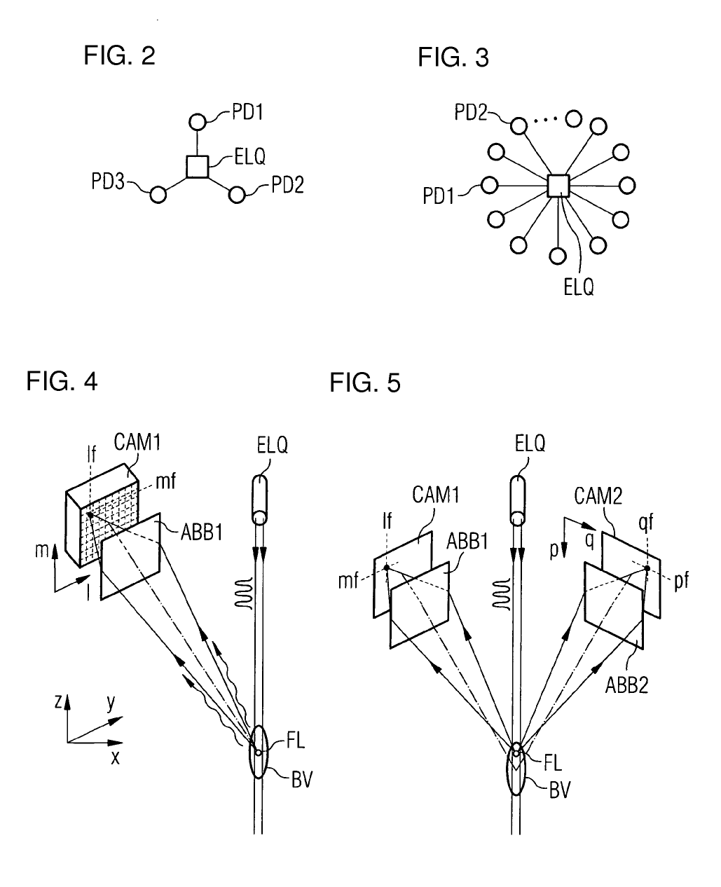

FIG. 2 shows a second apparatus according to the invention comprising three photodiodes,

FIG. 3 shows a third apparatus according to the invention, extended by a plurality of photodiodes,

FIG. 4 shows a fourth apparatus according to the invention comprising a camera,

FIG. 5 shows a fifth apparatus according to the invention comprising two cameras.

FIG. 1 shows an irradiation unit BA such as a linear accelerator, at least one beam output RAY thereof and/or beam axis thereof targeting a tumor TU1, TU2 to be destroyed (for example in the chest or lung region, but the extreme positions TU1, TU2 of the tumor could be at a different position of the body) of a person (not shown) lying on a table T. As a result of respiration or unexpected movements of the person, the tumor unavoidably moves relative to the pre-planned radiation position (i.e. isocenter) in the body, i.e. a movement volume is formed, which is defined by the extreme positions TU1, TU2 of the tumor. Before radiotherapy it is usual, for example, to perform a computed tomography scan in the chest or lung region, so that repositioning the apparatus EV according to the invention relative to a roughly estimated movement volume of the tumor is subsequently simplified. This assists an operator to position the apparatus according to the invention relative to the patient, but advantageously this positioning does not have be carried out in a highly accurate manner, it being at least sufficient if a fluorescing radiation F1 is able to be determined, which takes place after switching on an extrinsic light source ELQ for exciting a fluorophore FL arranged on the tumor, and in that a measuring signal of the excitation is determined in at least one optoelectric transducer PD1 of the apparatus EV according to the invention.

When the extrinsic light source ELQ of the apparatus EV, for example in the form of a wave with high frequency pulses, is switched on, when this wave strikes the fluorophore FL a fluorescing excitation from the fluorophore introduced into the tumor is produced in the form of a back-scattered wave F1. This wave F1 is finally determined by the optical recording apparatus PD1, selectively according to the wavelength. In other words, a mono-dimensional distance measurement between the apparatus EV according to the invention and the fluorophore FL moving with the tumor is possible according to the principle of a light-forming “echo” (i.e. transit time measurement). The light source ELQ and the photodiode may also be parts of an interferometer for measuring distances. To this end, therefore, a phase shifting method or white light interferometry, for example, is used, a degree of coherency between the light from the light source ELQ and the light from the fluorophore being intended to be ensured, in order to ensure that is able to interfere with one another. Thus a mono-dimensional localization of the moving tumor TU1, TU2 may take place in a coordinate system X, Y, Z, which, for example, is a coordinate system fixed to the table T. A positioning adjuster and/or measurer POS knows the position of the table T relative to the beam RAY of the irradiation unit BA, as well as the position of the apparatus EV according to the invention. In other words, the position of the tumor is now also able to be calculated at a determined distance of any point (for example of the isocenter) of the coordinate system X, Y, Z by means of a computer R. To this end, the computer R in combination with the irradiation unit, is connected to the positioning unit POS and possibly to the apparatus EV according to the invention, for example for triggering the measurements or for processing the measuring results. The positioning unit controls and determines the movements and/or positions of the apparatus EV according to the invention, of the table T and possibly of the irradiation unit BA. From the computer R an alarm signal may be triggered, if a discrepancy is established between a determined reference isocenter and the current actual isocenter. On the other hand, other signals may be triggered, in order to incorporate rapidly the beam path RAY of the irradiation unit BA at the determined reference isocenter. Naturally, the object is to carry out this procedure at a high frequency, so that the actual isocenter always coincides with the moving tumor.

For the further (two- or) three-dimensional localization of a movable tumor TU1, TU2 in a movement volume BV the apparatus EV according to the invention may also only have the individual photodiodes PD1, but in this case they should be mounted on, for example, a swivel mount, so that, for example, for a three-dimensional localization of the tumor via its coordinates XK, YK, ZK in the coordinate system X, Y, Z at least three measurements may be carried out for three different positions of the photodiode.

A first solution consists in moving at least the photodiode PD1 and/or the entire apparatus EV according to the invention on a track BAHN in a spatially known step (a minimum of three times) and to carry out a measurement for each step. This track BAHN should always be arranged to the side of beam paths emitted from the linear accelerator BA, even when these beam paths might be set in motion. The extrinsic light source ELQ may be positioned on this track BAHN and possibly travel therewith.

A second solution consists in rotating the photodiode PD1 about the optical axis of the extrinsic light source ELQ in a spatially known step (a minimum of 3 times). To this end, a simple rotating device ROT may be used, so that the apparatus EV according to the invention may be rotated.

Alternatively, the optical input axis of the individual photodiode PD1 (and/or the optical transit paths between the tumor and photodiode) could be spatially altered, for example by the use of switching elements (mirrors, prisms, etc.) between the photodiode PD1 and the tumor TU1, TU2. These switchable elements should ideally form a known pivoting of the optical axis of the photodiode PD1 about the isocenter.

In FIG. 2 the apparatus EV shown according to the invention is expanded by two further photodiodes PD2, PD3, which are arranged adjacent to the first photodiode PD1 and with one another. Thus, a threefold, i.e. a three-dimensional, distance measurement may be carried out, without for example moving or pivoting a photodiode and/or the apparatus EV according to the invention. An apparatus comprising two photodiodes PD1, PD2 would also be possible but it requires at least one further movement (see ROT in FIG. 1) or pivoting (see BAHN in FIG. 1, in order to determine a three-dimensional position of the tumor.

In FIG. 3 a further embodiment is disclosed, which forms a development according to FIG. 2. More than three photodiodes PD1, PD2, etc. are arranged in the space next to the extrinsic light source ELQ. The position of each photodiode is known in the coordinate system X, Y, Z according to FIG. 1, so that in the coordinate system X, Y, Z, absolute distance measurements between each photodiode and the fluorophore are always possible. A few of the photodiodes may be arranged at different distances from the light source ELQ or/and from the patient (i.e. from the fluorophore), so that for example low and high amplitudes of the measuring signals on the photodiodes (according to the amount of backlight from the fluorophore) are determined by a flexible measuring dynamic.

FIG. 4 now shows a further embodiment according to FIG. 1, instead of the photodiode PD1 a camera CAM1 such as a CCD camera being arranged in the apparatus EV according to the invention with adjacent pixels in an imaging plane m, l (i.e. optoelectric transducers). The fluorophore is imaged in the imaging plane m, l of the camera CAM1, via an optical imaging device ABB1 with a focal depth selected to be sufficient due to the movement of the fluorophore. At the same time, an aperture of the optical imaging device ABB1 should be opened so that the focal depth is ensured over the movement volume BV, but also not selected to be too small, so that light losses are avoided on the camera CAM1. This aspect is essential, depending on how deep a tumor is located in the body, as the light reflected from a deep fluorophore (for example in the lungs) is absorbed more than the light from a superficial tumor (for example on the eyes or on the face). Now, by this geometric optical imaging, which may be metrically calibrated in the coordinate system X, Y, Z, two spatial coordinates (for example X, Y) may be determined within the imaging plane m, l by means of the coordinates lf, mf of the fluorophore. The apparatus EV according to the invention, therefore, allows a two-dimensional localization of the tumor in the coordinate system X, Y, Z. For the three-dimensional localization of the fluorophore according to FIG. 1 the camera CAM1 may be moved/pivoted at fixed positions. Moreover, a wavelength-selective filter may be arranged in the optical imaging device ABB1 in order to keep extraneous light away from the fluorophore or/and a camera is used, the pixel technology thereof having highly sensitive recording properties in the spectral range of the light returning from the fluorophore.

In FIG. 5 a development of the apparatus EV according to the invention according to FIG. 4 is disclosed, in which a camera CAM2 is arranged in addition to the camera CAM1, so that the optical axes of the cameras CAM1, CAM2 are oriented in the direction of the body part with the fluorophore and the movement volume thereof and strike the movement volume at a positive angle (the optical axes form an individual, common point of intersection, the distance thereof being determined by the fluorophore/tumor). In other words, the cameras CAM1, CAM2 are triangulated within one plane, which should be roughly positioned in the vicinity of the movement volume. According to the embodiment, it is now possible to derive from FIG. 4 that both optical imaging devices ABB1, ABB2 for the two triangulating cameras CAM1, CAM2 deliver two geometrically imaged two-dimensional positions mf, if and pf, of the fluorophore/tumor from which the three-dimensional coordinates of the fluorophore/tumor may be calculated in the final coordinate system X, Y, Z (see FIG. 1) by means of a computer or a pre-programmed image memory. Thus a simple and rapid, three-dimensional localization of the tumor is ensured. The two optical imaging devices ABB1, ABB2 are adjusted such that a punctiform image of the fluorophore/tumor is taken on each camera, the lateral resolution thereof being sufficient, for example between 1/10 and 1/100 of the measuring range.

A further embodiment of the apparatus according to the invention EV would also be possible by further cameras being arranged in addition to the two cameras CAM1, CAM2 such that their optical axes meet at a separate point of intersection. Thus the localization of the tumor may be carried out more accurately and rapidly.

More generally, the optical recording apparatus PD1, PD2 . . . etc and/or CAM1, CAM2, . . . or/and the light source ELQ may also be arranged on a positioning device as a previous circular arc-shaped mount which positions the optical recording apparatus or/and the light source to the side of the being, such that light paths are minimized between, on the one hand, optical inputs of the optical recording apparatus and/or optical outputs of the light source and, on the other hand, of the body part and/or the movement volume thereof. As a result, it is ensured that a reduced amplitude of the measuring signals from the fluorophore is determined. Thus, in particular with a constant amount of energy from the extrinsic light source ELQ, lower metered quantities of the fluorophore are required or deeper tumors (which hardly radiate) may be visualized in the body.

In a complementary or alternative manner, the light source may have a bundled beam output, the energy distribution and energy density thereof being adjusted along a transverse surface of the beam output, such that reflection and/or backscatter of the fluorophore is measured by a sufficient signal-noise interval on the optical recording apparatus. This is particularly suitable for low reflective tumors or when a tumor sinks to different depths in the body due to its motion path in the body, but has to be still visible by fluorescence at all the different depths.

In this case, the bundled beam output may also have a longitudinal main axis, which may be pivoted by means of a high-frequency oscillating element for scanning the movement volume of the body part. Thus the bundled energy of the extrinsic light source ELQ may be transmitted in a more concentrated manner at allocated locations of the movement volume and form a grid pattern in the movement volume (surface) of the tumor by rapid scanning. This also avoids a burning effect on the skin and/or soft tissue parts of the patient, as the energy only remains very briefly at the same point of the illuminated skin/soft tissue parts. The light source may also emit periodic, pulsed light signals in these directions.

As already mentioned, the optical recording apparatus and/or the optical input of a photodiode/CCD camera have filters for the spectral isolation of reflection and/or backscatter of the fluorophore.

It may also be the case that the irradiation unit interferes with the electronics of the optical recording apparatus. One solution consists in that the input of the optical recording apparatus is guided via a waveguide (glass fiber or bundle of glass fibers) from the surface of the patient to the optical transducers arranged further away. Thus reflection and/or backscatter of the fluorophore is optimally transmitted, and by screening the glass fibers light components which do not belong to the fluorescence do not penetrate/interfere with the recording apparatus according to the invention. In other words, the electronically interfering components of the optical recording apparatus are removed from the radiation RAY (see FIG. 1), with the addition of an interposed optical waveguide.

It is also advantageous to consider that the photodiodes and/or the CCD cameras do not require a high resolution, but rather good measuring dynamics as a result of sensitive pixels which are as wide as possible (optoelectric transducers). As a result of large pixels, a greater amount of light is recorded, which is very advantageous for tumors positioned deep in the body, as a large amount of light from the fluorophore in the body is absorbed/damped, and thus will hardly reach the optical recording apparatus.

It is also provided in the invention that the optical recording apparatus is connected to a computer unit with at least one image memory and a processor unit, in which by means of recorded data (for example amplitude values of spatially known pixels) of the optical recording apparatus and by means of a detectable position of the optical recording device, relative to a known three-dimensional coordinate system X, Y, Z, three-dimensional coordinates XK, YK, ZK of the body part (tumor) may be determined in the coordinate system X, Y, Z in real time. The computer unit may, if required, be connected to a control module for the calculable repositioning of the apparatus according to the invention relative to the body part, for example in order to seek maximum amplitude values of specific pixels of the optical recording apparatus by sequential movement of the apparatus, when the patient lies on the table before treatment. As the movement or position of the apparatus may be determined relative to the coordinate system X, Y, Z, by means of a metric calibration of the optical components of the optical recording apparatus, the three-dimensional position of the illuminating fluorophore (i.e. of the tumor) may be established permanently and accurately, so that by repositioning the patient the tumor may also be permanently and accurately irradiated.

With the invention, it is possible for unhealthy cells to be located in the head, i.e. on the eyes or on the face, or in the lung or chest region of the person. In other words, the apparatus may be used very generally for the whole body. The energy from the light source only has to be altered according to the amount of fluorescence back-scattered from the fluorophore.

The use of the apparatus forms a navigation method per se or for further planning, observation or therapeutic treatment. In particular, the determinable three-dimensional coordinates XK, YK, ZK of the body part are used for controlling an irradiation unit of the body part or to assist a three-dimensional imaging system of the body or to assist a therapeutic planning tool.

For the use of the apparatus, moreover, in a simple manner the fluorophore may:
have a tumor affinity and be injected into a vein, preferably in the form of hematoporphyrin, or
be applied in an encapsulated form at a location of the body part, for example during a biopsy or during an endoscopic procedure, or
be deposited on a flat lozenge which is applied in the region of the surface of the body part.

It has also been shown that a use of the apparatus exhibits good results, in which, in the excited state, the fluorophore transmits light waves in the spectral range 600-760 nm, when the extrinsic light source emits light ideally in the spectral range 450-770 nm or at least pulsed laser light with a wavelength of 532 nm and thus excites the fluorophore.

The apparatus according to the invention also has a very advantageous use, in which it is a measuring head for controlling a mechanism for repositioning the being in an absolute coordinate system. In real time, the apparatus may output metric data, by which for example the table with the patient is repositioned relative to the beam path of an irradiation unit. Thus the position of the tumor (i.e. the fluorophore) continuously and accurately coincides with the isocenter of the radiation center.

1-17. (canceled)

18. An apparatus for the spatial localization of a moveable body part situated inside a movement volume on a surface or extending as far as inside a living being, the apparatus comprising:
at least one optical recording apparatus arranged outside the living being;
at least one meterable fluorophore for introduction in a region of the body part;
an extrinsic radiation source disposed outside the living being and generating radiation propagating in a direction of the movement volume, for causing a spectral excitation of the fluorophore, wherein a wave emitted by the fluorophore is produced and can be determined at a wavelength measured by said optical recording apparatus;
said optical recording apparatus having at least one optical axis which can be oriented in the direction of the body part and the movement volume thereof;
the optical recording apparatus having at least one optoelectric transducer disposed perpendicularly to said optical axis and outputting an output signal for determining a distance between the fluorophore and a reference point.

19. The apparatus according to claim 18, wherein said optoelectric transducer of said optical recording apparatus is a photodiode or a group of mutually adjacent photodiodes or pixels.

20. The apparatus according to claim 19, wherein said optoelectric transducer of said optical recording apparatus includes a group of mutually adjacent pixels of a camera sensor.

21. The apparatus according to claim 19, wherein said optical recording apparatus is movably disposed, such that the optical axes formed thereby enclose a positive angle with one another and form a separate point of intersection in the movement volume.

22. The apparatus according to claim 21, wherein said optical recording apparatus is movably disposed for pivoting and/or rotation.

23. The apparatus according to claim 18, wherein said optical recording apparatus comprises a plurality of photodiodes or a plurality of cameras with mutually adjacent optoelectric transducers, the optical axes thereof forming a positive angle with one another and forming a separate point of intersection in the movement volume.

24. The apparatus according to claim 19, wherein said optical recording apparatus and/or said radiation source are disposed on a positioning device configured to position said optical recording apparatus and/or said radiation source to a side of the living being, such that light paths are minimized between optical inputs of said optical recording apparatus and/or optical outputs of said radiation source, on the one hand, and of the body part and/or the movement volume thereof on the other hand.

25. The apparatus according to claim 18, wherein said radiation source is a light source with a bundled beam output having an energy distribution and energy density adjusted along a transverse surface of the beam output, such that reflection and/or backscatter of the fluorophore is measured by a sufficient signal-noise interval on the optical recording apparatus.

26. The apparatus according to claim 18, which comprises at least one optical waveguide disposed between an output of said radiation source and the movement volume.

27. The apparatus according to claim 18, which comprises at least one optical waveguide disposed between an input of said optical recording apparatus and the movement volume.

28. The apparatus according to claim 18, which comprises an oscillating switching element disposed in a light path between said extrinsic radiation source and said optical recording apparatus.

29. The apparatus according to claim 18, wherein said optical recording apparatus includes filters for a spectral isolation of reflection and/or backscatter of the fluorophore.

30. The apparatus according to claim 18, wherein said optical recording apparatus is connected to a computer unit, and said computer unit is configured to determine in real time three-dimensional coordinates of the body part in a three-dimensional coordinate system from recorded data of the optical recording apparatus and by way of a detectable position of the optical recording apparatus relative to the three dimensional coordinate system.

31. The apparatus according to claim 30, wherein the optical data are maximized amplitude values obtained from the fluorophore.

32. The apparatus according to claim 18, wherein the body part comprising the fluorophore is formed of unhealthy cells and the apparatus is configured to determine three-dimensional coordinates thereof in real time relative to a known three-dimensional coordinate system.

33. The apparatus according to claim 32, wherein the unhealthy cells are located in the head, or in the lung, or chest region of a person.

34. The apparatus according to claim 32, wherein the unhealthy cells are disposed in the eyes or on the face of a person.

35. The apparatus according to claim 32, wherein the three-dimensional coordinates of the body part are used for controlling an irradiation unit of the body part or to assist a three-dimensional imaging system of the body or to assist a therapeutic planning tool.

36. The apparatus according to claim 32, wherein the fluorophore:
has a tumor affinity and is injected into a vein; or
is applied in an encapsulated form at a location of the body part; or
is deposited on a flat lozenge applied in the region of the surface of the body part.

37. The apparatus according to claim 36, wherein the fluorophore is hematoporphyrin injected into a vein.

38. The apparatus according to claim 36, wherein the fluorophore is applied in an encapsulated form during a biopsy or during an endoscopic procedure.

39. The apparatus according to claim 32, wherein the fluorophore is configured to transmit light waves in a spectral range of 600-760 nm in an excited state, when the extrinsic radiation source emits light ideally in a spectral range of 450-770 nm or pulsed laser light with a wavelength of 532 nm for exciting the fluorophore.

40. The apparatus according to claim 18, wherein the apparatus is a measuring head for controlling a mechanism for repositioning the living being in an absolute coordinate system.
